(19) United States
(12) Patent Application Publication
(10) Pub. No.: US 2007/0156174 A1
(43) Pub. Date: Jul. 5, 2007

(54) Method and apparatus for repairing a meniscus

(75) Inventors: Ryan A. Kaiser, Leesburg, IN (US); Kevin T. Stone, Winona Lake, IN (US)

Correspondence Address: HARNESS, DICKEY & PIERCE, P.L.C., P.O. BOX 828, BLOOMFIELD HILLS, MI, 48303, US

(73) Assignee: Arthrotek, Inc., Warsaw, IN (US)

(21) Appl. No.: 11/324,367

(22) Filed: Jan. 3, 2006

(51) Int. Cl. A61B 17/08; A61D 1/00

(52) U.S. Cl. 606/215

(57) ABSTRACT

An apparatus for repairing a separation in body tissue includes an elongated repair member having an intermediate portion extending between a first end and a second end. A first retaining portion is defined at the first end. A second retaining portion is defined at the second end. The elongated repair member is porous and adapted to allow vascularization through the body tissue while urging opposing portions of the separation together in an implanted position.

### FIELD

This invention relates generally to a method and apparatus for use in repairing soft tissue, and more particularly, to a method and apparatus for repairing a torn meniscus during arthroscopic surgery.

### BACKGROUND

There are many techniques employed to repair damaged soft tissue. These techniques include suturing, stapling, taping and the like. Selection of which technique to employ depends upon the type of soft tissue being repaired, the soft tissue location and the required strength of the repair. While there exists many techniques to repair soft tissue, there is a growing need to easily and quickly repair a torn meniscus in the knee during arthroscopic surgery.

The meniscus tissue is a fibrocartilaginous structure in the knee joint which performs multiple critical functions, including contributing to normal knee biomechanics and the general well-being of the joint. Generally, the menisci are comprised of two C-shaped fibrocartilaginous structures residing on the tibial plateau. The peripheral rim of a meniscus is thick, tapering to a thin, free inner border. The superior surface is concave to contact the femoral condyles, while the inferior surface is flat to contact the tibial plateau. The fibers forming the menisci are mainly oriented circumferentially throughout the meniscus, parallel to the peripheral border, to withstand hoop stresses placed upon the meniscus by the femoral condyles.

A peripheral region or zone of the meniscus is generally referred to as a red/red zone that promotes good blood supply. A central region or zone of the meniscus is generally referred to as a white/white zone that is avascular. An intermediate region or zone is generally referred to as red/white that has variable blood supply. It is generally recognized that repair of meniscal lesions, to the extent possible, is preferable to excision so as to attempt to maintain the normality of the meniscus and have it continue to function as intended. In addition, it is important to maintain vascularity within the peripheral area and intermediate area of the meniscus to promote healing.

### SUMMARY

An apparatus for repairing a separation in body tissue includes an elongated repair member having an intermediate portion extending between a first end and a second end. A first retaining portion is defined at the first end. A second retaining portion is defined at the second end. The elongated repair member is porous and adapted to allow vascularization through the body tissue while urging opposing portions of the separation together in an implanted position.

According to various features, the first and second retaining portions are adapted to engage each other. In another embodiment, the first retaining portion is integrally formed with the elongated repair member.

According to other features, the elongated repair member may be resorbable or non-resorbable collagen.

A method for repairing a separation in body tissue includes forming a passage in the body tissue at a location to repair the separation, the passage defining an entrance and an exit. The repair member is passed through the passage. The repair member is manipulated whereby opposing surfaces of the separation are engaged. Passage of bodily fluid is enabled through the repair member to promote healing at the separation.

According to other features, the passage is formed from the entrance at a first area of the body tissue, through the tear and out the exit at a second area of the body tissue. The first end of the repair member is advanced into the tissue at the entrance. The second end of the repair member is pulled away from the entrance whereby opposing surfaces of the separation are urged together.

Further areas of applicability of the present disclosure will become apparent from the detailed description provided hereinafter. It should be understood that the detailed description and various examples, while indicating various embodiments of the invention, are intended for purposes of illustration only and are not intended to limit the scope of the following claims.

### BRIEF DESCRIPTION OF THE FIGURES

The present invention will become more fully understood from the detailed description and the accompanying drawings, wherein:

FIG. 1A is a perspective view of a meniscus repair apparatus according to the present teachings;

FIG. 1B is a perspective view of a meniscus repair apparatus according to additional features;

FIG. 1C is a perspective view of a meniscus repair apparatus according to additional features;

FIG. 1D is a perspective view of a meniscus repair apparatus according to additional features;

FIG. 2A is a perspective view of a meniscus repair apparatus having an integral retaining feature according to the present teachings;

FIG. 2B is a perspective view of a meniscus repair apparatus having an integral retaining feature according to additional features;

FIG. 2C is a perspective view of a meniscus repair apparatus having an integral retaining feature according to additional features;

FIG. 2D is a perspective view of a meniscus repair apparatus having an integral retaining feature according to additional features;

FIG. 3 is a perspective view of a torn meniscus;

FIG. 4 illustrates the meniscus repair apparatus of FIG. 1A in an implanted position;

FIG. 5 illustrates the meniscus repair apparatus of FIG. 1D in an implanted position;

FIG. 6 illustrates a meniscus repair apparatus having the integral retaining feature illustrated in FIG. 2A shown in an implanted position;

FIG. 7 illustrates the meniscus repair apparatus of FIG. 2B shown in an implanted;

FIG. 8 illustrates the meniscus repair apparatus of FIG. 2C shown in an implanted position;

FIG. 9 illustrates the meniscus repair apparatus of FIG. 2D shown in an implanted position;

FIG. 10A illustrates a meniscus repair apparatus according additional features; and

FIG. 10B illustrates the meniscus repair apparatus of FIG. 10A shown in an implanted position.

### DETAILED DESCRIPTION OF VARIOUS EMBODIMENTS

The following description of various embodiment(s) is merely exemplary in nature and is in no way intended to limit the application or uses.

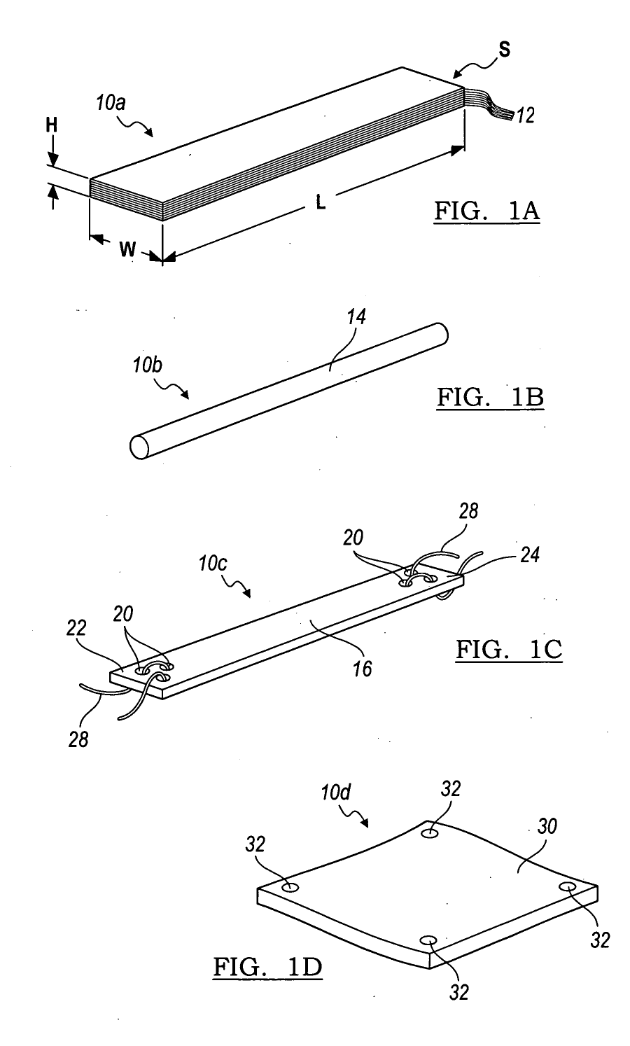

With initial reference to FIG. 1A, an apparatus for repairing a tear in meniscal tissue is shown generally at reference 10a. The apparatus 10a includes a multiple layer section of material. As shown in FIG. 1A, the section of material is illustrated as generally rectangular in shape and defines a length L, width W and a height H. While illustrated as eight layers along the height H, additional or fewer layers may be employed. As will become appreciated from the following discussion, the multiple layer section of material defines a scaffold S that may be shaped into a number of configurations to reinforce and repair a meniscal tear site. It is appreciated however, that while the following discussion is specifically directed to repairing a tear in a meniscus, the same may be applied to other body tissues. Likewise, while the specific examples herein are directed to a tear, the same may be applied to other tissue separations such as, but not limited to, incisions, breaks and cuts.

In general, for tissue to heal, adequate blood flow must be permitted in the subject area. A suitable material according to an embodiment of the present teachings may comprise multiple layers 12 of intestinal collagen. The collagen may be acellular crosslinked collagen. In one form, the collagen is resorbable and may have low crosslinking to allow for remodeling. The material properties of collagen is porous and facilitates blood flow in the vascular zones of the meniscus to promote healing. In one example, the collagen may have a porosity from 5% to 60%. Other resorbable and non-resorbable materials may be employed.

With reference to FIGS. 1B-1D, other embodiments of the apparatus are shown and referred to generally at 10b-10d. While not specifically illustrated as having multiple layers, the remaining embodiments may comprise either a single layer or multiple layers as depicted in relation to the apparatus 10a in FIG. 1A. The apparatus 10b is configured as a cylindrical body 14. The apparatus 10c in FIG. 1C defines a body portion 16 having a plurality of pre-formed apertures 20 at opposite ends 22 and 24. The apertures 20 may accept sutures 28 as needed for fastening. The apparatus 10d illustrated in FIG. 1D is configured generally as a square patch 30. The patch 30 may have apertures 32 for locating fastening devices such as sutures for example.

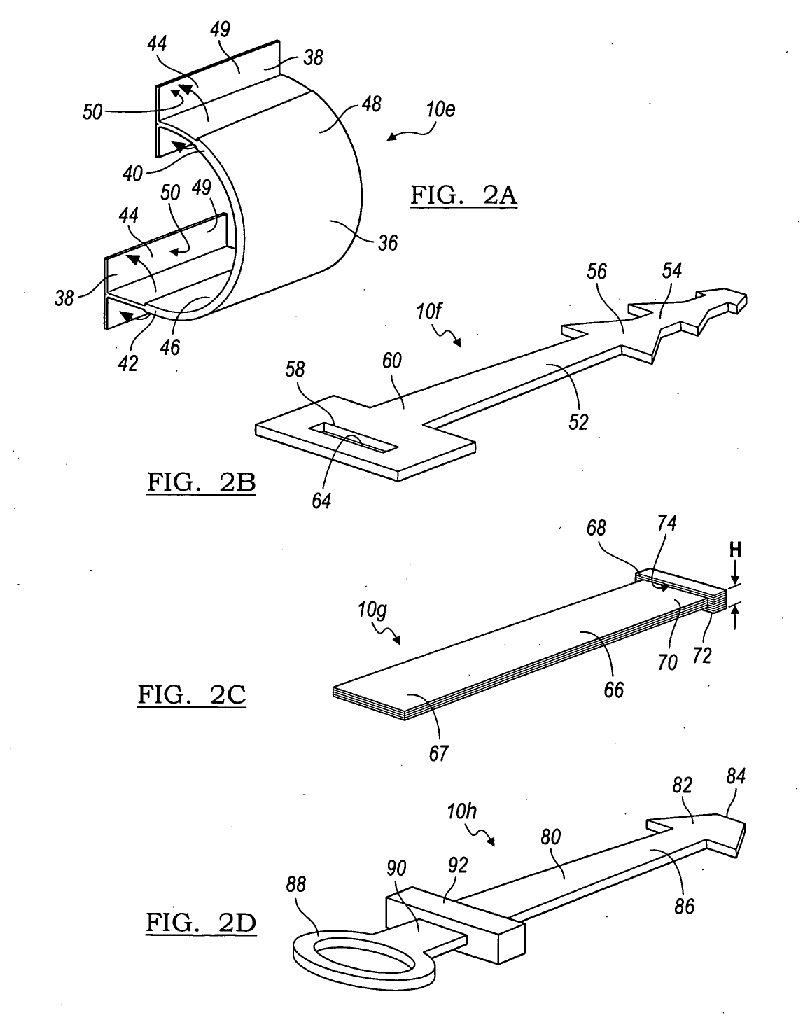

With reference now to FIGS. 2A-2D, other embodiments of the apparatus are shown and referred to generally at 10e-10h. With initial reference to the apparatus 10e shown in FIG. 2A, a main body portion-36 is shown having integrally formed retaining portions 38 defined on opposite ends 40 and 42. In one example, the integrally formed retaining portions 38 define flap portions 44. The flap portions 44 may be created by cutting a section of material at a front surface 46 and at a rear surface 48, respectively, near the ends 40 and 42 of the main body portion 36. The sections, once cut, may be rotated outwardly to form the flap portions 44. As a result, the flap portions 44 are integral to the main body portion 36. The flap portions 44 each define a planar portion 49 having an engagement face 50. As will be described, the engagement face 50 is adapted to engage and press against an outer tissue surface in an implanted position. The flap portions 44 may be created by other methods.

Turning now to FIG. 2B, the apparatus 10f according to another embodiment is shown. The apparatus 10f generally defines a main body 52 having an angled tooth portion 54 at a distal end 56 and a loop portion 58 at a proximal end 60. As will be described in greater detail herein, a passage 64 defined through the loop portion 58 is adapted to receive the tooth portion 54 in an implanted position.

With reference to FIG. 2C, the apparatus 10g is shown. The apparatus 10g generally comprises a main body portion 66 at first free end 67 and an integrally formed retaining portion 68 defined on a second end 70. In one example, the apparatus may be formed from the scaffold S illustrated in FIG. 1A. In this regard, the integrally formed retaining portion 68 may comprise a plurality of layers of material defining a height H, such as illustrated in relation to the apparatus 10 in FIG. 1A. The main body 66 may be formed by removing a layer or layers of material along the length L, width W and/or height H from the scaffold S. Similar to the apparatus 10e illustrated in FIG. 2A, the integrally formed retaining portion 68 defines a planar portion 72 having an engagement face 74. As will be described, the engagement face 74 is adapted to engage and press against an outer tissue surface in an implanted position.

With reference now to FIG. 2D, the apparatus 10h is shown. The apparatus 10h generally includes a main body portion 80 having a toothed portion 82 defining a point 84 formed on a distal end 86 and a loop portion 88 formed on a proximal end 90. A retaining block 92 is defined on the proximal end 90 near the loop portion 88. In one example, the features of the apparatus 10h may be formed from the scaffold S illustrated in FIG. 1A. As a result, the apparatus 10h defines integrally formed features. As will be described, the apparatus 10h is adapted to locate tissue between the toothed portion 82 and the retaining block 92 in an installed position.

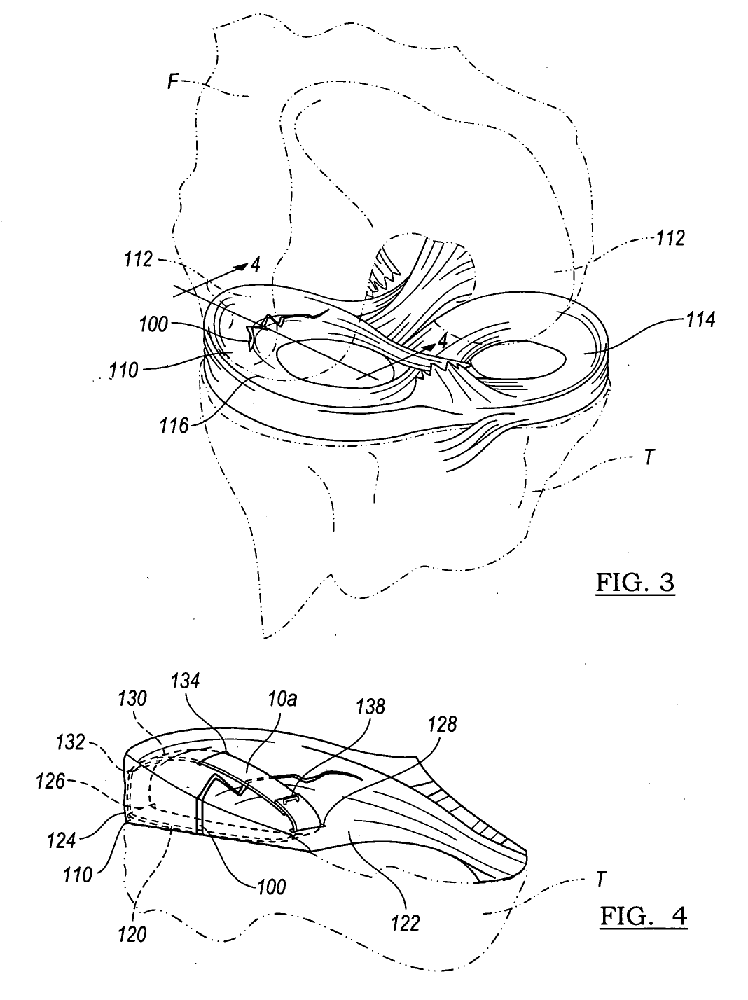

Turning now to FIG. 3, a posterior view of a human knee represented by a tibia T and femur F in phantom is shown having a tear 100 in a meniscus 110. For exemplary purposes, the knee is a right knee although it is appreciated that the same principles apply to a left knee. The femur F generally defines a pair of condyles 112 which rest on a medial meniscus 114 and a lateral meniscus 116. While the tear 100 is shown on a periphery of the lateral meniscus 116, it is appreciated that the various apparatuses disclosed herein may be adapted to repair a tear located elsewhere on the meniscus 110.

The following FIGS. 4-9 are taken along line 4-4 of FIG. 3 and illustrate various apparatus in an implanted position. FIG. 4 illustrates the apparatus of FIG. 1A shown in an implanted position. In one exemplary method, a first path 120 is formed, such as by a knife or other sharp instrument, between a first surface 122 to a second surface 124. The first path 120 is defined between openings 126 and 128 and transcends the tear 100. A second path 130 may be formed between the first and second surfaces 122 and 124. The second path 130 is defined between openings 132 and 134. Once the respective paths 120 and 130 are formed, opposite ends of the apparatus 10a are secured to the meniscus 110 at the first surface 122 such as by way of staples 138. Of note, a first portion of the apparatus 10a transcends through the tear 100 within the meniscus 110 while a second portion of the apparatus 10a transcends the tear 100 across the first surface 122 of the meniscus 110. It is appreciated that the second path 130 is optional and the apparatus 10a may simply wrap around an outer perimeter of the meniscus 110 or be contained entirely within the meniscus 110. Again, as mentioned above, the material properties of the collagen encourages blood flow in the vascular zones of the meniscus 110 to promote healing.

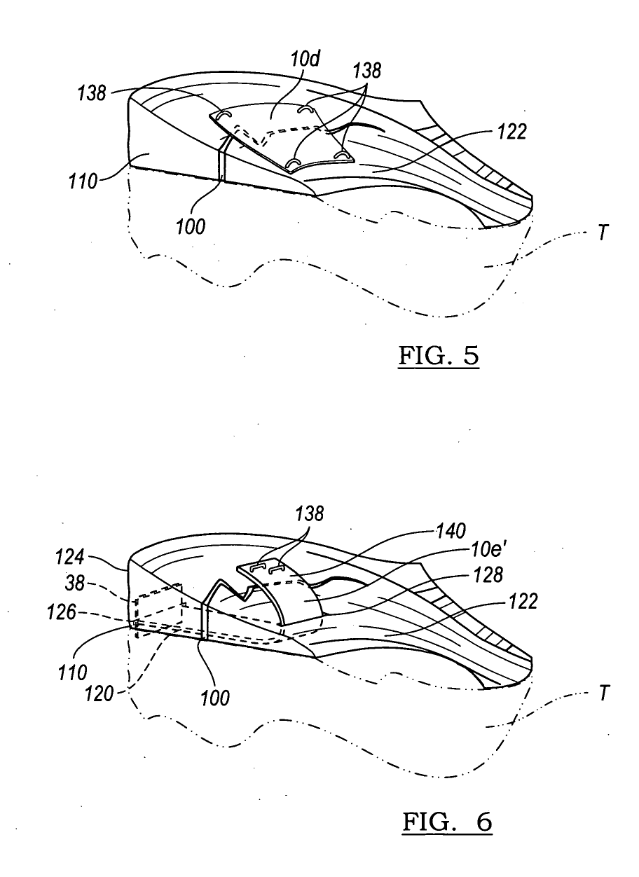

FIG. 5 illustrates the apparatus 10d of FIG. 1D shown in an implanted position. In one exemplary method, the apparatus 10d is positioned to transcend across the tear 100 in the meniscus 110. The apparatus 10d is then secured to the first surface 122 of the meniscus 110 such as by staples 138. In one application, the apparatus 10d is secured such that opposing meniscal tissue along the tear 100 is placed in compression.

With reference now to FIG. 6, a version of the apparatus 10e illustrated in FIG. 2A is shown in an implanted position and generally referenced at 10e′. The integral retaining portion 38 is shown formed on one end of the apparatus 10e′. A free end 140 is defined opposite the retaining portion 38. In one exemplary method, a path 120 is formed, such as by a knife or other sharp instrument, between a first surface 122 to a second surface 124. The path 120 is defined between openings 126 and 128 and transcends the tear 100. Once the apparatus 10e is pulled to place opposing meniscal tissue along the tear 100 in compression, the free end 140 of the apparatus 10e may then be secured to the first surface 122 of the meniscus 110 such as by staples 138.

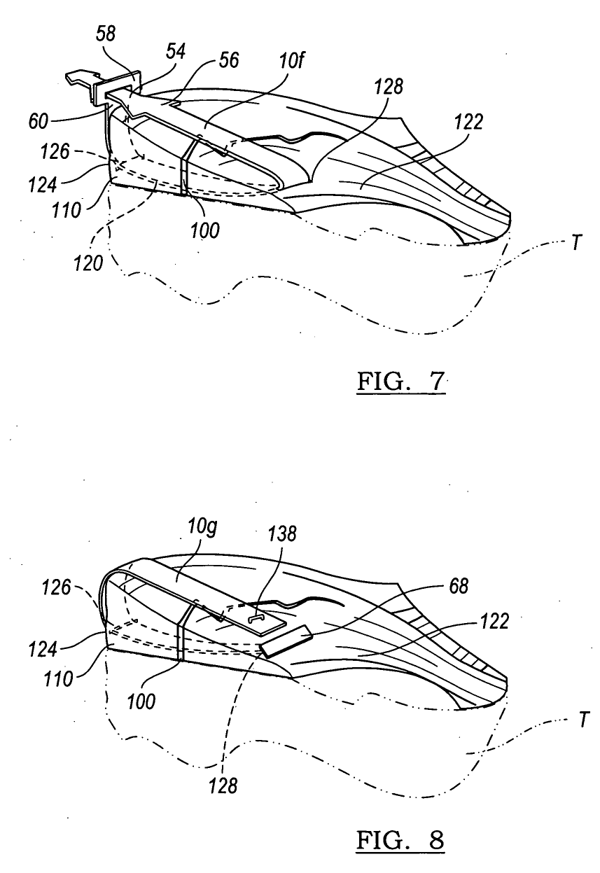

Turning now to FIG. 7, the apparatus 10f of FIG. 2B is shown in an implanted position. In one exemplary method, a path 120 is formed, such as by a knife or other sharp instrument, between a first surface 122 to a second surface 124. The path 120 is defined between openings 126 and 128 and transcends the tear 100. Once the apparatus 10f is passed through the path 120, the angled tooth portion 54 at the distal end 56 is inserted through the loop portion 58 at the proximal end 60. The distal end 56 is then pulled until the loop portion 58 nests behind a tooth of the toothed portion 54 in a secure position. Any excess material at the proximal end 56 may subsequently be cut off if desired. Alternatively, the apparatus 10f may be contained entirely within the meniscus 110

With reference now to FIG. 8, the apparatus 10g illustrated in FIG. 2C is shown in an implanted position. In one exemplary method, a path 120 is formed, such as by a knife or other sharp instrument, between a first surface 122 and a second surface 124. The path 120 is defined between openings 126 and 128 and transcends the tear 100. The apparatus 10g is then located through the path 120 until the integral retaining structure 68 engages the first surface 122. Once the apparatus 10g is pulled to place opposing meniscal tissue along the tear in compression, the free end of the apparatus 10g may then be secured to a surface 122 of the meniscus such as by staples 138.

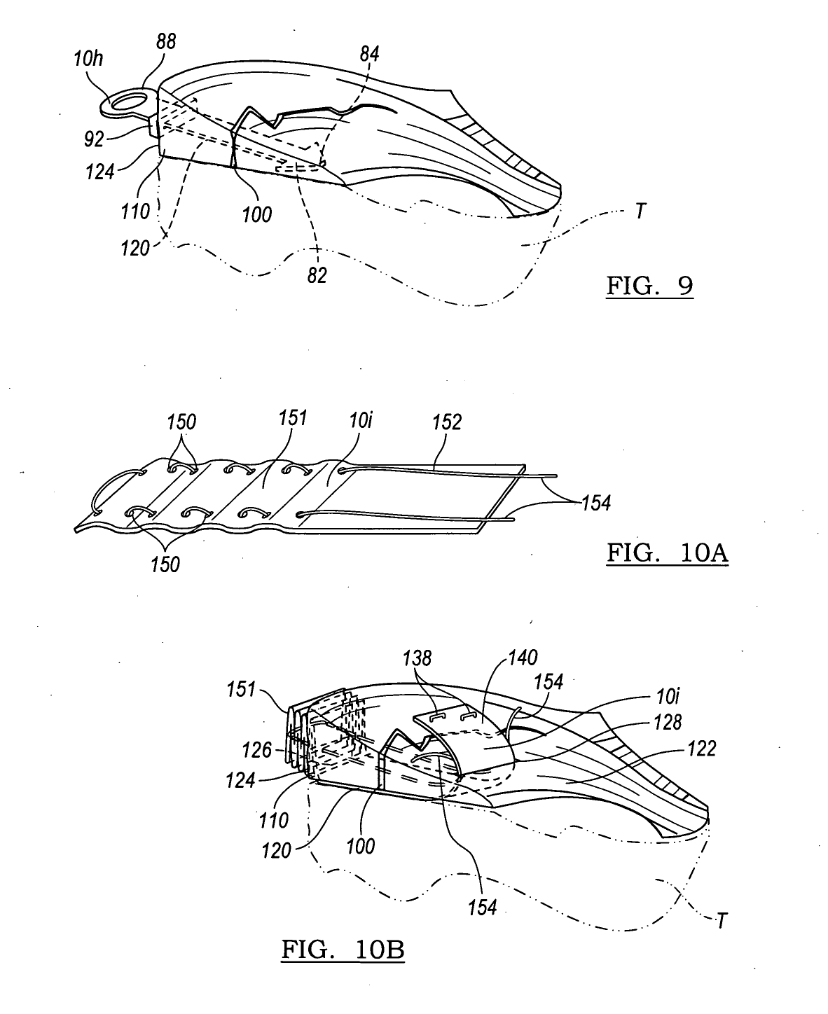

Turning now to FIG. 9, the apparatus 10h of FIG. 2D is shown in an implanted position. In one exemplary method, a path 120 is formed, such as by a knife or other sharp instrument from a peripheral surface 124 to a location within the meniscus 110. The path 120 transcends the tear 100. The apparatus 10h is then inserted through the path 120 until the toothed portion 82 passes beyond the tear 100. It is appreciated that the width of the path 120 is less than the span of the toothed portion 82. The loop portion 88 is subsequently pulled away from the meniscus 110 causing the toothed portion 82 to pull the meniscus toward its perimeter and as a result, placing the opposing meniscal tissue along the tear 100 in compression. The looped portion 88 may subsequently be cut off if desired. In another exemplary method, the point 84 of the toothed portion 82 may be used to form the path 120 through the meniscus 110. In this way, a surgeon may translate the apparatus 10h along its longitudinal axis while the point 84 of the toothed portion 82 pierces the meniscus 110 until reaching a location beyond the tear 100.

FIGS. 10A and 10B illustrate an accordion style apparatus 10i according to an additional embodiment. The apparatus 10i may be formed of one or multiple layers of the scaffold S. A plurality of apertures 150 are defined at a first end 151 around a perimeter of the apparatus 10i. A suture 152 is passed through the respective apertures 150 and defines a pair of free ends 154. Once the apparatus 10i is passed through a prepared passage 120, such as described above, the free ends 154 of the suture 152 may be translated such that the first end 151 of the apparatus 10i bunches up in a wave-like pattern. As a result, the first end 151 forms retaining structure for engaging an outer surface of the meniscus 110 when implanted. A second end 160 may be secured to-a surface 122 of the meniscus 110 such as by staples 138.

Those skilled in the art can now appreciate from the foregoing description that the broad teachings of the present invention can be implemented in a variety of forms. For example, while the preceding discussion explains first, forming a passage in the tissue and subsequently passing the apparatus through the passage, the same may be accomplished simultaneously. In one example, a hollow piercing member may carry the apparatus and concurrently form the passage while depositing the apparatus through the passage. Furthermore, while some examples illustrate repairing a meniscal tear by securing the apparatus on the outside of the meniscus or passing the apparatus through a passage in the meniscus having opposite ends secured to the outside of the meniscus, the apparatus may be contained entirely within the meniscus. In addition, while staples 138 have been shown for securing the various apparatus, other securing methods may be employed, such as but not limited to sutures, such as sutures 152-illustrated in FIG. 10A. Therefore, while this invention has been described in connection with particular examples thereof, the true scope of the invention should not be so limited since other modifications will become apparent to the skilled practitioner upon a study of the drawings, the specification and the following claims.

What is claimed is:

1. An apparatus for repairing a separation in body tissue comprising:
an elongated repair member having an intermediate portion extending between a first end and a second end;
a first retaining portion defined at said first end;
a second retaining portion defined at said second end; and
wherein said elongated repair member is porous and adapted to allow vascularization through the body tissue while urging opposing portions of the separation together in an implanted position.

2. The apparatus of claim 1 wherein said first and second retaining portions are adapted to engage each other.

3. The apparatus of claim 2 wherein said first retaining portion is integrally formed with said elongated repair member.

4. The apparatus of claim 3 wherein at least one of said first and second retaining portions are attached to the tissue by a supplemental device.

5. The apparatus of claim 4 wherein said supplemental device comprises at least one of staples and sutures.

6. The apparatus of claim 3 wherein said integrally formed retaining portion defines a planar portion having an engagement face, said engagement face adapted to press against the body tissue in said implanted position.

7. The apparatus of claim 6 wherein said elongated repair member defines a first width along said intermediate portion and a second width along said planar portion and wherein said first and second widths are substantially the same.

8. The apparatus of claim 1 wherein said elongated repair member is resorbable.

9. The apparatus of claim 6 wherein said elongated repair member comprises collagen.

10. The apparatus of claim 9 wherein said elongated repair member comprises acellular crosslinked intestinal collagen.

11. The apparatus of claim 1 wherein said elongated repair member defines passages formed therein.

12. A method of repairing a separation in body tissue comprising:
forming a passage in the body tissue at a location to repair the separation, the passage defining an entrance and an exit;
passing a repair member through the passage;
manipulate the repair member whereby opposing surfaces of the separation are engaged; and
enabling passage of bodily fluid through the repair member to promote healing at the separation.

13. The method of claim 12 wherein forming the passage comprises:
forming the passage from the entrance at a first area of the body tissue, through the tear and out the exit at a second area of the body tissue.

14. The method of claim 10 wherein manipulating the elongated piece of material further comprises:
advancing a first end of the repair member into the tissue at the entrance; and
pulling a second end of the repair member away from the entrance whereby opposing surfaces of the separation are urged together.

15. The method of claim 10 wherein manipulating the repair member comprises:
advancing a second end of the repair member out of the exit until an engagement face defined on a first end provides a contact force a surface of the tissue.

16. The method of claim 15 wherein manipulating the repair member further comprises:
at least one of stapling and suturing the second end of the repair member to a surface of the tissue.

17. An apparatus for repairing a separation in body tissue comprising:
an elongated repair member having an intermediate portion extending between a first end and a second end, the first end adapted to be secured to a first area of the soft tissue in an implanted position;
an integrally formed retaining portion positioned at said second end, said integrally formed retaining portion is adapted to engage a second area of the soft tissue; and
wherein said elongated repair member is porous and adapted to allow vascularization through the body tissue while urging opposing portions of the separation together in an implanted position.

18. The apparatus of claim 17 wherein said integrally formed retaining portion defines a planar portion having an engagement face, said engagement face adapted to press against said second surface in said implanted position.

19. The apparatus of claim 18 wherein said repair member defines a first width along said intermediate portion and said second end defines a second width along said planar portion, wherein said first and second widths are substantially the same.

20. The apparatus of claim 17 wherein said elongated repair member is resorbable.

21. The apparatus of claim 20 wherein said elongated repair member comprises collagen.

22. The apparatus of claim 21 wherein said elongated repair member comprises acellular crosslinked intestinal collagen.

23. The apparatus of claim 17 wherein said elongated repair member defines passages formed therein.
